(19) United States
(12) Patent Application Publication
(10) Pub. No.: US 2008/0227168 A1
(43) Pub. Date: Sep. 18, 2008

(54) METHODS AND MATERIALS FOR EXTRA AND INTRACELLULAR DELIVERY OF CARBON NANOTUBES

(75) Inventors: Tanja Kmecko, Plano, TX (US); Gareth Hughes, Irving, TX (US); Rockford K Draper, Plano, TX (US); Robert M. Burgess, Garland, TX (US); Austin David-Edmiston Swafford, Richardson, TX (US)

Correspondence Address: CHALKER FLORES, LLP, 2711 LBJ FRWY, Suite 1036, DALLAS, TX, 75234, US

(73) Assignees: BOARD OF REGENTS, THE UNIVERSITY OF TEXAS SYSTEM, Austin, TX (US); MEDICAL NANOTECHNOLOGIES, INC., Richardson, TX (US)

(21) Appl. No.: 12/032,665

(22) Filed: Feb. 16, 2008

Related U.S. Application Data
(60) Provisional application No. 60/902,007, filed on Feb. 16, 2007.

(51) Int. Cl. C12N 13/00; C12N 5/00; C08G 75/00; C08G 73/08

(52) U.S. Cl. 435/173.1; 435/375; 528/373; 528/401; 528/403; 528/423; 528/392; 977/746

(57) ABSTRACT

The present invention includes compositions and methods to deliver carbon nanostructures that include agents for delivery to cells, wherein the carbon nanostructure and the agent are made soluble by coating the carbon nanostructure with one or more polymers, e.g., low band gap conductive polymers.

### CROSS-REFERENCE TO RELATED APPLICATIONS

This application claims priority to U.S. Provisional Application Ser. No. 60/902,007, filed Feb. 16, 2007, the entire contents of which are incorporated herein by reference.

### TECHNICAL FIELD OF THE INVENTION

The present invention relates in general to the field of nanomedicine, and more particularly, to compositions and methods for highly effective targeting of nanostructures to target cells.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with.

Functionalized carbon nanotubes (CNTs) are emerging as new tools in the field of nanobiotechnology and nanomedicine due to the fact that they can be easily manipulated and modified by encapsulation with biopolymers or by covalent or non-covalent functionalization. Carbon nanotubes have optical, electronic, and mechanical properties that can be exploited in biological or biomedical applications including, but not limited to, cancer therapeutics. CNT absorption of electromechanical radiation and radiofrequency (RF), near infrared radiation (near IR) and visible light (VIS) results in efficient thermal heating which may be implemented for biomedical applications. The possibility of incorporating CNTs into biological systems has provided new opportunities for exploration of their potential applications in biology and medicinal chemistry, but they are hydrophobic and difficult to interface with living organisms (Banerjee et al., (2003) Chemistry: 9 1898-1908).

Thus, the use and application of carbon nanotubes in a biological setting depends on developing agents that can adapt the surface of carbon nanotubes to the aqueous biological environment. Conjugated polymers or oligomers can be designed to tightly interact with CNTs in a non-covalent fashion resulting in a significant reduction of CNT-CNT van der Waals interactions. The minimization of van der Waals interactions plays a role in effective CNT dispersion which is inherently required for the introduction of CNTs to a biological setting. In addition, as conductive polymers are based upon an electron-deficient aromatic system that makes their backbone positively charged, one advantage of their use for biological applications is strong electrostatic interaction with the negatively charged cell membrane. Further, the ability to customize polymer or oligomer side chains allows for effective solubility in an aqueous environment as well as the efficient attachment of targeting moieties such as antibodies, peptides, carbohydrates, folate and folate receptors and other small molecules. The attachment of therapeutics may also be accomplished through these side chains thus resulting in the development of combination therapies. The use of water-soluble conjugated systems for the aqueous dispersion of carbon nanotubes coupled with cell specific targeting capabilities represents new opportunities for biological and biomedical applications such as cancer therapy.

### SUMMARY OF THE INVENTION

More particularly, the present invention includes compositions, methods and systems for intra and extracellular delivery of compositions to cells comprising by contacting one or more cells with a compositions comprising a water-soluble rod-coil polymer, wherein the water-soluble rod-coil polymer comprises an agent for delivery to the cell about a carbon nanostructure, wherein the water-soluble rod-coil polymer is coated with a water-soluble, low band gap conductive polymer. In one aspect, the carbon nanostructures are carbon nanotubes. In another aspect, the agent is within the carbon nanostructure. In another aspect, the agent is water-insoluble, susceptible to degradation by an organism when administered systemically, or highly toxic to the host when administered systemically.

In one aspect of the present invention, the water-soluble rod-coil polymer has a solubility of greater than 20 times that of the non-coated carbon nanostructure. For example, the intracellular uptake of the water-soluble rod-coil polymer can be measured with normal rat kidney cells, and the water-soluble rod-coil polymer achieves the same level of intracellular location as a non-polymer coated carbon nanostructure at less than one twentieth the concentration of water-soluble rod-coil polymers. The methods may also include the step of contacting the water-soluble rod-coil polymer with cells, wherein the water-soluble rod-coil polymer is capable of intracellular transport at a concentration at least 10 times lower that carbon nanostructures that are non-polymer coated. In another aspect, the method may also include the step of contacting the water-soluble rod-coil polymer with cells, wherein the water-soluble rod-coil polymer is capable of intracellular transport at a concentration at least 10 times lower that carbon nanostructures that are non-polymer coated and disrupting the water-soluble rod-coil polymer within the cells to deliver the agent. In yet another aspect, the carbon nanostructure includes carbon nanotubes coated with poly(3,4-ethylenedioxythiophene) poly(styrenesulfonate) (PEDOT/PSS).

In another embodiment, the present invention includes a water-soluble conjugated system that includes a water-soluble rod-coil polymer having a carbon nanostructure coated with a water-soluble, low band gap conductive polymers selected from polypyrroles (PPy), polythiophene (PT), poly(3,4-ethylenedioxythiophene) poly(styrenesulfonate) (PEDOT/PSS); water-soluble low band gap polymer such as: poly(thieno[3,4-b]thiophenes), poly(thienylene vinylenes), hydroxyl-capped polyfluorenes (PFOH), cyclopentadithiophenes (CPDT) and oligomers and alternating copolymers thereof, ionic water-soluble polymers (cationic or anionic), polyfluorenes (PF), alternating polyfluorenylene ethynylene (PFE) copolymers comprising alternating fluorene and phenylene-oxadiazole-phenylene comprising alternating fluorene and 1,4-phenylene; water-soluble linear polyindolquinones; water-soluble poly(p-phenylenes) (PPP); water-soluble poly(p-phenylene vinylene) (PPV); water-soluble polypyridines; water-soluble ionic polyacetylene; and a biologically active agent disposed about the carbon nanostructure and the polymer. In one aspect, the polymer is selected from poly(thienylene vinylenes), cyclopentadithiophenes (CPDT), polyfluorenes (PF), polyfluorenylene ethynylene (PFE), phenylene-oxadiazole-phenylene, Polyindolquinones, and polypyridines. In another aspect, the polymer is selected from polypyrroles (PPy), polythiophene (PT), poly(3,4-ethylenedioxythiophene) poly(styrenesulfonate) (PEDOT/PSS), poly(thieno[3,4-b]thiophenes), hydroxyl-capped polyfluorenes (PFOH), 1,4-phenylene, poly(p-phenylenes) (PPP), poly(p-phenylene vinylene) (PPV) and polyacetylene.

In another aspect of the present invention, the dispersed carbon nanostructure includes carbon nanotubes that are further functionalized through the addition of a targeting moiety. For example, the carbon nanostructure comprises carbon nanotubes further comprising a targeting moiety selected from antibody, aptamer, peptide, protein, carbohydrate, lipid, nucleic acid, folate, folate receptor or a small molecule. In another aspect, the carbon nanostructure may also include a liposome surrounding the water-soluble rod-coil polymer. In one example, the carbon nanostructure is a carbon nanotubes coated with poly(3,4-ethylenedioxythiophene) poly(styrenesulfonate) (PEDOT/PSS) and the water-soluble rod-coil polymer is capable of intracellular transport at a concentration at least 10 times lower that carbon nanostructures that are not coated with a polymer. In certain aspects, the water-soluble rod-coil polymers are targeted to the nucleus of cells.

In yet another aspect of the invention, the water-soluble rod-coil polymers are targeted to the nucleus of cells and include an agent that modulates gene expression. In another aspect, the water-soluble rod-coil polymer is targeted to the cytoplasm of cells and comprises an agent that modulates cellular function. The water-soluble rod-coil polymer may be further disrupted using a radiofrequency field targeted to the carbon nanostructure or an infrared radiation that disrupts the carbon nanostructure. In another aspect, the water-soluble rod-coil polymer is disrupted to release the agent and the agent is capable of killing the cell. In yet another aspect, the water-soluble rod-coil polymer includes one or more agents that are highly toxic, degraded by the host or are provided as a reservoir for controlled release by an external source of radiation.

In another embodiment, the present invention includes a method of killing target cells by contacting cells with a water-soluble rod-coil polymer comprising a carbon nanostructure coated with a water-soluble, low band gap conductive polymer under conditions in which the cells bind or internalize the water-soluble rod-coil polymer; and exposing the target cells with the water-soluble rod-coil polymer with sufficient near infrared or radiofrequency radiation to kill the cell by hyperthermia.

### BRIEF DESCRIPTION OF THE DRAWINGS

For a more complete understanding of the features and advantages of the present invention, reference is now made to the detailed description of the invention along with the accompanying figures and in which:

FIG. 1 is a scanning electron microscope (SEM) image of single wall carbon nanotubes (SWNTs) dispersed in water via the conductive polymer PEDOT/PSS and evaporated into a thin film.

FIG. 2 is a scanning electron microscope (SEM) image of multi-wall carbon nanotubes (MWNTs) dispersed in water via PEDOT/PSS and evaporated into a thin film. MWNTs are evident as singulated fibers.

FIG. 3 is an optical image of normal rat kidney cells with the intracellular presence of single wall carbon nanotubes dispersed and functionalized by PEDOT/PSS.

FIG. 4 is a diagrammatic illustration of CNT-polymer-targeting moiety system wherein the targeting moiety is an antibody.

FIGS. 5A to 5C show NRK cells incubated in medium for 2 days at 37° C. with or without SWNTs dispersed in the medium.

FIGS. 6A to 6b show NRK cells exposed to SWNTs coated with PEDOT/PSS.

FIG. 7A shows NRK cells were exposed to SWNTs (approximately 5 μg/ml) dispersed in PEDOT/PSS for 2 days. FIG. 7B shows a Raman spectra from the vesicle showing the distinctive SWNT Raman signature.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “agents,” “active ingredient(s),” “pharmaceutical ingredient(s),” “active agents,” “bioactive agent” are defined as drugs and/or pharmaceutically active ingredients. The present invention may be used to encapsulate, attach, bind or otherwise be used to affect the storage, stability, longevity and/or release of any of the following drugs as the pharmaceutically active agent in a composition.

One or more of the following bioactive agents may be combined with the water-soluble rod-coil polymer and one or more carriers and the present invention (which may itself be the carrier): analgesic anti-inflammatory agents such as, acetaminophen, aspirin, salicylic acid, methyl salicylate, choline salicylate, glycol salicylate, 1-menthol, camphor, mefenamic acid, fluphenamic acid, indomethacin, diclofenac, alclofenac, ibuprofen, ketoprofen, naproxene, pranoprofen, fenoprofen, sulindac, fenbufen, clidanac, flurbiprofen, indoprofen, protizidic acid, fentiazac, tolmetin, tiaprofenic acid, bendazac, bufexamac, piroxicam, phenylbutazone, oxyphenbutazone, clofezone, pentazocine, mepirizole, and the like.

Drugs having an action on the central nervous system, for example sedatives, hypnotics, antianxiety agents, analgesics and anesthetics, such as, chloral, buprenorphine, naloxone, haloperidol, fluphenazine, pentobarbital, phenobarbital, secobarbital, amobarbital, cydobarbital, codeine, lidocaine, tetracaine, dyclonine, dibucaine, cocaine, procaine, mepivacaine, bupivacaine, etidocaine, prilocalne, benzocaine, fentanyl, nicotine, and the like.

Antihistaminics or antiallergic agents such as, diphenhydramine, dimenhydrinate, perphenazine, triprolidine, pyrilamine, chlorcyclizine, promethazine, carbinoxamine, tripelennamine, brompheniramine, hydroxyzine, cyclizine, meclizine, clorprenaline, terfenadine, chlorpheniramine, and the like. Anti-allergenics such as, antazoline, methapyrilene, chlorpheniramine, pyrilamine, pheniramine, and the like.

Decongestants such as, phenylephrine, ephedrine, naphazoline, tetrahydrozoline, and the like.

Antipyretics such as, aspirin, salicylamide, non-steroidal anti-inflammatory agents, and the like. Antimigrane agents such as, dihydroergotamine, pizotyline, and the like.

Acetonide anti-inflammatory agents, such as hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone, betamethasone, ibuprophen, naproxen, fenoprofen, fenbufen, flurbiprofen, indoprofen, ketoprofen, suprofen, indomethacin, piroxicam, aspirin, salicylic acid, diflunisal, methyl salicylate, phenylbutazone, sulindac, mefenamic acid, meclofenamate sodium, tolmetin, and the like.

Steroids such as, androgenic steriods, such as, testosterone, methyltestosterone, fluoxymesterone, estrogens such as, conjugated estrogens, esterified estrogens, estropipate, 17-β estradiol, 17-β estradiol valerate, equilin, mestranol, estrone, estriol, 17β ethinyl estradiol, diethylstilbestrol, progestational agents, such as, progesterone, 19-norprogesterone, norethindrone, norethindrone acetate, melengestrol, chlormadinone, ethisterone, medroxyprogesterone acetate, hydroxyprogesterone caproate, ethynodiol diacetate, norethynodrel, 17-α hydroxyprogesterone, dydrogesterone, dimethisterone, ethinylestrenol, norgestrel, demegestone, promegestone, megestrol acetate, and the like.

Respiratory agents such as, theophilline and β₂-adrenergic agonists, such as, albuterol, terbutaline, metaproterenol, ritodrine, carbuterol, fenoterol, quinterenol, rimiterol, solmefamol, soterenol, tetroquinol, and the like.

Sympathomimetics such as, dopamine, norepinephrine, phenylpropanolamine, phenylephrine, pseudoephedrine, amphetamine, propylhexedrine, arecoline, and the like.

Local anesthetics such as, benzocaine, procaine, dibucaine, lidocaine, and the like.

Antimicrobial agents including antibacterial agents, antifungal agents, antimycotic agents and antiviral agents; tetracyclines such as, oxytetracycline, penicillins, such as, ampicillin, cephalosporins such as, cefalotin, aminoglycosides, such as, kanamycin, macrolides such as, erythromycin, chloramphenicol, iodides, nitrofrantoin, nystatin, amphotericin, fradiomycin, sulfonamides, purroInitrin, clotrimazole, miconazole chloramphenicol, sulfacetamide, sulfamethazine, sulfadiazine, sulfamerazine, sulfamethizole and sulfisoxazole; antivirals, including idoxuridine; clarithromycin; and other anti-infectives including nitrofurazone, and the like.

Antihypertensive agents such as, clonidine, α-methyldopa, reserpine, syrosingopine, rescinnamine, cinnarizine, hydrazine, prazosin, and the like. Antihypertensive diuretics such as, chlorothiazide, hydrochlorothrazide, bendoflumethazide, trichlormethiazide, furosemide, tripamide, methylclothiazide, penfluzide, hydrothiazide, spironolactone, metolazone, and the like. Cardiotonics such as, digitalis, ubidecarenone, dopamine, and the like. Coronary vasodilators such as, organic nitrates such as, nitroglycerine, isosorbitol dinitrate, erythritol tetranitrate, and pentaerythritol tetranitrate, dipyridamole, dilazep, trapidil, trimetazidine, and the like. Vasoconstrictors such as, dihydroergotamine, dihydroergotoxine, and the like. β-blockers or antiarrhythmic agents such as, timolol pindolol, propranolol, and the like. Humoral agents such as, the prostaglandins, natural and synthetic, for example PGE₁, PGE₂α, and PGF₂α, and the PGE₁ analog misoprostol. Antispasmodics such as, atropine, methantheline, papaverine, cinnamedrine, methscopolamine, and the like.

Calcium antagonists and other circulatory organ agents, such as, aptopril, diltiazem, nifedipine, nicardipine, verapamil, bencyclane, ifenprodil tartarate, molsidomine, clonidine, prazosin, and the like. Anti-convulsants such as, nitrazepam, meprobamate, phenyloin, and the like. Agents for dizziness such as, isoprenaline, betahistine, scopolamine, and the like. Tranquilizers such as, reserprine, chlorpromazine, and antianxiety benzodiazepines such as, alprazolam, chlordiazepoxide, clorazeptate, halazepam, oxazepam, prazepam, clonazepam, flurazepam, triazolam, lorazepam, diazepam, and the like.

Antipsychotics such as, phenothiazines including thiopropazate, chlorpromazine, triflupromazine, mesoridazine, piperracetazine, thioridazine, acetophenazine, fluphenazine, perphenazine, trifluoperazine, and other major tranqulizers such as, chlorprathixene, thiothixene, haloperidol, bromperidol, loxapine, and molindone, as well as, those agents used at lower doses in the treatment of nausea, vomiting, and the like.

Muscle relaxants such as, tolperisone, baclofen, dantrolene sodium, cyclobenzaprine.

Drugs for Parkinson's disease, spasticity, and acute muscle spasms such as levodopa, carbidopa, amantadine, apomorphine, bromocriptine, selegiline (deprenyl), trihexyphenidyl hydrochloride, benztropine mesylate, procyclidine hydrochloride, baclofen, diazepam, dantrolene, and the like. Respiratory agents such as, codeine, ephedrine, isoproterenol, dextromethorphan, orciprenaline, ipratropium bromide, cromglycic acid, and the like. Non-steroidal hormones or antihormones such as, corticotropin, oxytocin, vasopressin, salivary hormone, thyroid hormone, adrenal hormone, kallikrein, insulin, oxendolone, and the like.

Vitamins such as, vitamins A, B, C, D, E and K and derivatives thereof, calciferols, mecobalamin, and the like for dermatologically use. Enzymes such as, lysozyme, urokinaze, and the like. Herb medicines or crude extracts such as, Aloe vera, and the like.

Examples of agents that are highly toxic when administered systemically include antitumor agents such as, 5-fluorouracil and derivatives thereof, krestin, picibanil, ancitabine, cytarabine, and the like. Anti-estrogen or anti-hormone agents such as, tamoxifen or human chorionic gonadotropin, and the like. Miotics such as pilocarpine, and the like.

Cholinergic agonists such as, choline, acetylcholine, methacholine, carbachol, bethanechol, pilocarpine, muscarine, arecoline, and the like. Antimuscarinic or muscarinic cholinergic blocking agents such as, atropine, scopolamine, homatropine, methscopolamine, homatropine methylbromide, methantheline, cyclopentolate, tropicamide, propantheline, anisotropine, dicyclomine, eucatropine, and the like.

Mydriatics such as, atropine, cyclopentolate, homatropine, scopolamine, tropicamide, eucatropine, hydroxyamphetamine, and the like. Psychic energizers such as 3-(2-aminopropy)indole, 3-(2-aminobutyl)indole, and the like.

Antidepressant drugs such as, isocarboxazid, phenelzine, tranylcypromine, imipramine, amitriptyline, trimipramine, doxepin, desipramine, nortriptyline, protriptyline, amoxapine, maprotiline, trazodone, and the like.

Anti-diabetics such as, insulin, and anticancer drugs such as, tamoxifen, methotrexate, and the like.

Anorectic drugs such as, dextroamphetamine, methamphetamine, phenylpropanolamine, fenfluramine, diethylpropion, mazindol, phentermine, and the like.

Anti-malarials such as, the 4-aminoquinolines, alphaminoquinolines, chloroquine, pyrimethamine, and the like.

Anti-ulcerative agents such as, misoprostol, omeprazole, enprostil, and the like.

Antiulcer agents such as, allantoin, aldioxa, alcloxa, N-methylscopolamine methylsuflate, and the like. Antidiabetics such as insulin, and the like.

For use with vaccines, one or more antigens, such as, natural, heat-killer, inactivated, synthetic, peptides and even T cell epitopes (e.g., T cell epitopes obtained from the following proteins, GADE, DAGE and MAGE).

The agents mentioned above may be used in combination as required. Moreover, the above drugs may be used either in the free form or, if capable of forming salts, in the form of a salt with a suitable acid or base. If the drugs have a carboxyl group, their esters may be employed.

The acid mentioned above may be an organic acid, for example, methanesulfonic acid, lactic acid, tartaric acid, fumaric acid, maleic acid, acetic acid, or an inorganic acid, for example, hydrochloric acid, hydrobromic acid, phosphoric acid or sulfuric acid. The base may be an organic base, for example, ammonia, triethylamine, or an inorganic base, for example, sodium hydroxide or potassium hydroxide. The esters mentioned above may be alkyl esters, aryl esters, aralkyl esters, and the like.

When a drug different than an anesthetic agent is used the solvent selected is one in that the drug is soluble. In generally the polyhydric alcohol may be used as a solvent for a wide variety of drugs. Other useful solvents are those known to solubilize the drugs in question.

The bioactive may also be administered parenterally, intraperitoneally, intraspinally, or intracerebrally. Dispersions may be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

Sterile injectable solutions may be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient (i.e., the therapeutic compound) plus any additional desired ingredient from a previously sterile-filtered solution thereof.

The bioactive may be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a subject.

Aqueous compositions of the present invention inclurdes an effective amount of the agent associated with a nanoparticle, nanotube, nanofibril or nanoshell or chemical composition of the present invention dissolved and/or dispersed in a pharmaceutically acceptable carrier and/or aqueous medium. The present invention is particularly useful for the delivery of water insoluble agents or agents that may be degraded by the organisms to cells.

The biological material should be extensively dialyzed to remove undesired small molecular weight molecules and/or lyophilized for more ready formulation into a desired vehicle, where appropriate. The active compounds may generally be formulated for parenteral administration, e.g., formulated for injection via the intravenous, intramuscular, sub-cutaneous, intralesional, and/or even intraperitoneal routes. The preparation of an aqueous compositions that contain an effective amount of the nanostructure composition as an active component and/or ingredient will be known to those of skill in the art in light of the present disclosure. Typically, such compositions may be prepared as injectables, either as liquid solutions and/or suspensions; solid forms suitable for using to prepare solutions and/or suspensions upon the addition of a liquid prior to injection may also be prepared; and/or the preparations may also be emulsified.

The pharmaceutical forms suitable for injectable use include sterile aqueous solutions and/or dispersions; formulations including sesame oil, peanut oil and/or aqueous propylene glycol; and/or sterile powders for the extemporaneous preparation of sterile injectable solutions and/or dispersions. In all cases the form must be sterile and/or must be fluid to the extent that easy syringability exists. It must be stable under the conditions of manufacture and/or storage and/or must be preserved against the contaminating action of microorganisms, such as bacteria and/or fungi.

Solutions of the active compounds as free base and/or pharmacologically acceptable salts may be prepared in water suitably mixed with a surfactant, such as hydroxypropylcellulose. Dispersions may also be prepared in glycerol, liquid polyethylene glycols, and/or mixtures thereof and/or in oils. Under ordinary conditions of storage and/or use, these preparations contain a preservative to prevent the growth of microorganisms.

The nanostructure composition of the present invention may be formulated into a composition in a neutral and/or salt form. Pharmaceutically acceptable salts, include the acid addition salts (formed with the free amino groups of the protein) and/or that are formed with inorganic acids such as, for example, hydrochloric and/or phosphoric acids, and/or such organic acids as acetic, oxalic, tartaric, mandelic, and/or the like. Salts formed with the free carboxyl groups may also be derived from inorganic bases such as, for example, sodium, potassium, ammonium, calcium, and/or ferric hydroxides, and/or such organic bases as isopropylamine, trimethylamine, histidine, procaine and/or the like.

The carrier may also be a solvent and/or dispersion medium containing, for example, water, ethanol, polyol (for example, glycerol, propylene glycol, and/or liquid polyethylene glycol, and/or the like), suitable mixtures thereof, and/or vegetable oils. The proper fluidity may be maintained, for example, by the use of a coating, such as lecithin, by the maintenance of the required particle size in the case of dispersion and/or by the use of surfactants. The prevention of the action of microorganisms may be brought about by various antibacterial and/or antifungal agents, for example, parabens, chlorobutanol, phenol, sorbic acid, thimerosal, and/or the like. In many cases, it will be preferable to include isotonic agents, for example, sugars and/or sodium chloride. Prolonged absorption of the injectable compositions may be brought about by the use in the compositions of agents delaying absorption, for example, aluminum monostearate and/or gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle that contains the basic dispersion medium and/or the required other ingredients from those enumerated above. In the case of sterile powders for the preparation of sterile injectable solutions, the preferred methods of preparation are vacuum-drying and/or freeze-drying techniques that yield a powder of the active ingredient plus any additional desired ingredient from a previously sterile-filtered solution thereof. The preparation of more, and/or highly, concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and/or in such amount as is therapeutically effective. The formulations are easily administered in a variety of dosage forms, such as the type of injectable solutions described above, but drug release capsules and/or the like may also be employed.

For parenteral administration in an aqueous solution, for example, the solution should be suitably buffered if necessary and/or the liquid diluent first rendered isotonic with sufficient saline and/or glucose. These particular aqueous solutions are especially suitable for intravenous, intramuscular, subcutaneous and/or intraperitoneal administration. In this connection, sterile aqueous media that may be employed will be known to those of skill in the art in light of the present disclosure. For example, one dosage could be dissolved in 1 ml of isotonic NaCl solution and/or either added to 1000 ml of hypodermoclysis fluid and/or injected at the proposed site of infusion, (see for example, “Remington's Pharmaceutical Sciences” 15th Edition, pages 1035-1038 and/or 1570-1580). Some variation in dosage will necessarily occur depending on the condition of the subject being treated. The person responsible for administration will, in any event, determine the appropriate dose for the individual subject.

In addition to the compounds formulated for parenteral administration, such as intravenous and/or intramuscular injection, other pharmaceutically acceptable forms include, e.g., tablets and/or other solids for oral administration; liposomal formulations; time release capsules; and/or any other form currently used, including cremes.

One may also use nasal solutions and/or sprays, aerosols and/or inhalants in the present invention. Nasal solutions are usually aqueous solutions designed to be administered to the nasal passages in drops and/or sprays. Nasal solutions are prepared so that they are similar in many respects to nasal secretions, so that normal ciliary action is maintained. Thus, the aqueous nasal solutions usually are isotonic and/or slightly buffered to maintain a pH of 5.5 to 6.5. In addition, antimicrobial preservatives, similar to those used in ophthalmic preparations, and/or appropriate drug stabilizers, if required, may be included in the formulation.

Additional formulations that are suitable for other modes of administration include vaginal suppositories and/or suppositories. A rectal suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions, suspensions, tablets, pills, capsules, sustained release formulations and/or powders. In certain defined embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

The tablets, troches, pills, capsules and/or the like may also contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup of elixir may contain the active compounds sucrose as a sweetening agent methyl and/or propylparabens as preservatives, a dye and/or flavoring, such as cherry and/or orange flavor.

The examples of pharmaceutical preparations described above are merely illustrative and not exhaustive; the nanostructures of the present invention are amenable to most common pharmaceutical preparations.

The inability to effectively disperse and functionalize a carbon nanostrucutres, e.g., nanotubes (CNTs) has significantly hindered efforts at developing CNT-related applications in biology and the life sciences. The presently described invention circumvents these hindrances and provides a novel mechanism for both dispersing and functionalizing CNTs for use in a biological setting.

As used herein, a “water-soluble rod-coil polymer” is used to describe a water-soluble conjugated system that includes a carbon nanostructure, an agent, and a polymer that increases the solubility of the carbon nanostructure for the effective delivery of a water-soluble rod-coil polymer to cells. Examples of such polymers include, but are not limited to, alternating copolymer or oligomer platforms are used in this application as dispersing and functionalizing agents through the attachment of targeting moieties such as antibodies, peptides, carbohydrates, small molecules and aptamers for the specific delivery of CNTs to particular cell types for biomedical applications, an example of which would be cancer cell killing. In addition, water-soluble conjugated polymer, copolymer or oligomer platforms may be used as targeting carriers for other biomolecules, drugs and other agents used in biological and biomedical applications. The polymers are designed to interact tightly with CNTs in a non-covalent manner thus resulting in efficient dispersion. The water solubility of these polymers, copolymers and oligomers has been obtained through introducing ionic function to their side chains. Interaction of these polymers with molecules for biological targeting has been obtained through hydroxyl, ester, ether, amide or thiol linkages. The conjugation length in these polymeric, copolymeric and oligomeric structures can be varied to achieve desired properties for a variety of biological applications.

One embodiment of the present invention includes the water-soluble conjugated systems. A second embodiment of the present invention includes the water-soluble conjugated system/CNT complex. A third embodiment of the present invention includes the water-soluble conjugated system/CNT complex covalently coupled to a targeting moiety. A fourth embodiment of the present invention includes targeting of the water-soluble conjugated system/CNT/targeting moiety complex to specific cell types. A fifth embodiment of the present invention includes cell killing via radiofrequency (RF) exposure to cells binding or internalizing the water-soluble conjugated system/CNT/targeting moiety complex. A sixth embodiment of the present invention includes cell killing via infrared radiation exposure to cells binding or internalizing the water-soluble conjugated system/CNT/targeting moiety. In another aspect, two or more carbon nanostructure are used that are selected to be disrupted by two or more different radiofrequencies, wherein the two or more carbon nanostructures carry different agents, and wherein the agents are complementary, e.g., an anti-neoplastic agent and a potentiator of the anti-neoplastic agent.

The CNT/PEDOT/PSS highly soluble was made as follows. Briefly, PEDOT/PSS was dissolved in water to give 8.3 mg/ml of the polymer composite, and 10 mg of SWNTs were added to 10 ml of the aqueous polymer solution. The composition was then sonicated for at least 30 minutes. The SWNTs were CoMoCAT tubes from Southwest Nanotechnologies (www.swnano.com), PEDOT and PSS were purchased from Sigma-Aldrich (USA).

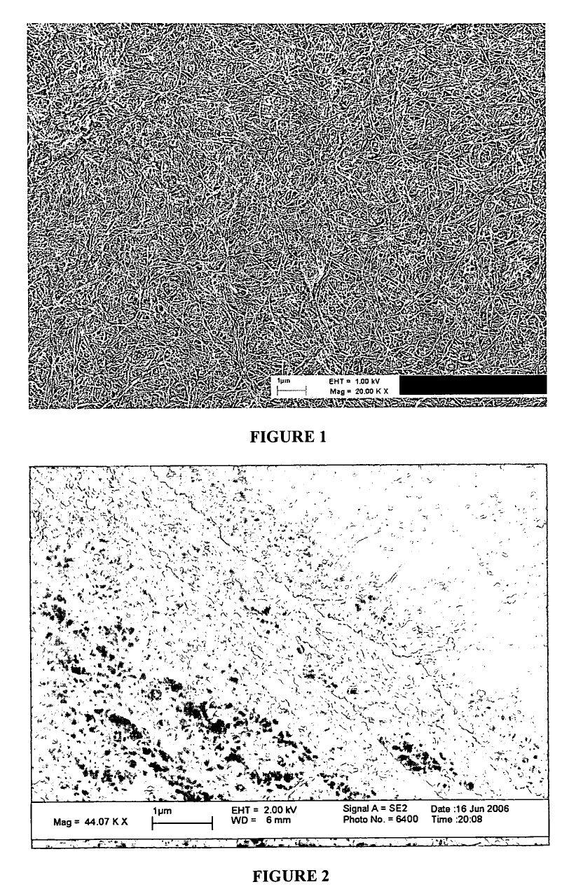

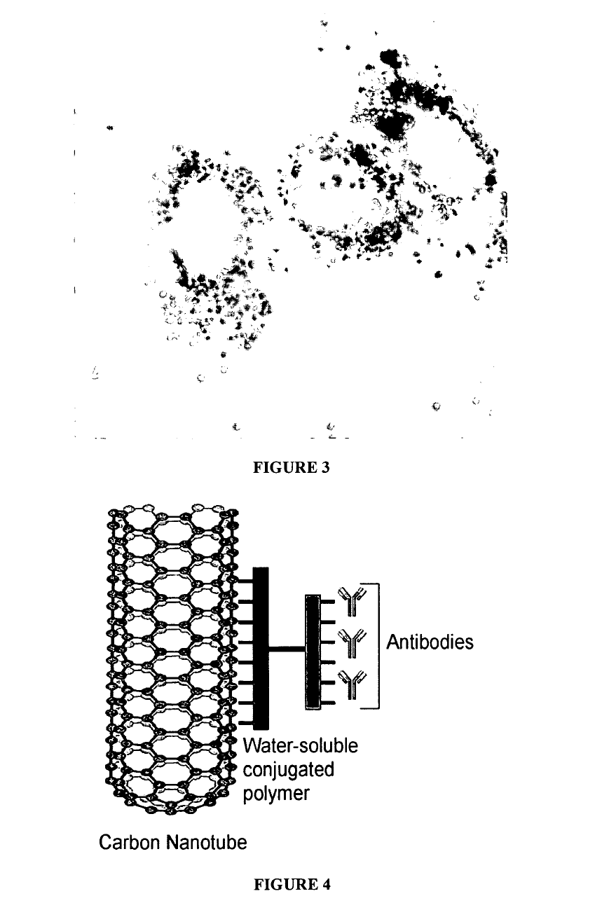

FIG. 1 is a scanning electron microscope (SEM) image of single wall carbon nanotubes (SWNTs) dispersed in water via the conductive polymer PEDOT/PSS and evaporated into a thin film. It was found that the SWNT-PEDOT/PSS nanotubes where highly soluble in water. A significant difference in solubility was visually observed between SWNT and the SWNT-PEDOT/PSS nanotubes. This increase in solubility was even noticeable under a light microscope, including when cells were imaged with the SWNT-PEDOT/PSS nanotubes (see FIG. 3 below).

FIG. 2 is a scanning electron microscope (SEM) image of multi-wall carbon nanotubes (MWNTs) dispersed in water via PEDOT/PSS and evaporated into a thin film. MWNTs are evident as singulated fiber-like structures.

FIG. 3 is an optical image of normal rat kidney cells with the intracellular presence of single wall carbon nanotubes dispersed and functionalized by PEDOT/PSS. Surprisingly, the SWNT-PEDOT/PSS nanotubes were visible within these normal rat kidney cells under the light microscope, confirming the significant increase in solubility observed for the SWNT-PEDOT/PSS nanotubes. Furthermore, the cellular uptake of SWNT-PEDOT/PSS nanotubes by these non-transformed cells was significant.

FIG. 4 is a diagrammatic illustration of CNT-polymer-targeting moiety system wherein the targeting moiety is an antibody.

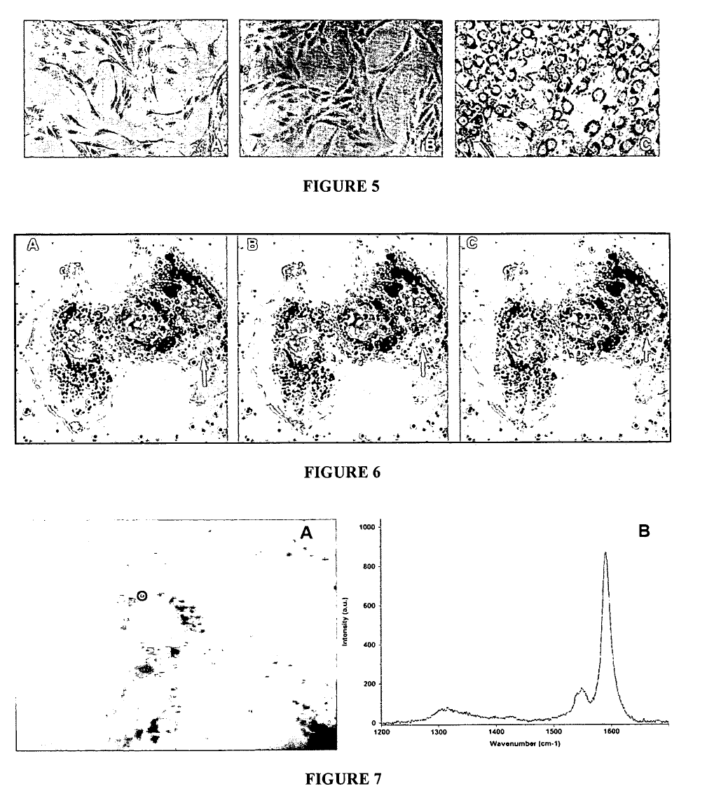

FIGS. 5A to 5C show NRK cells incubated in medium for 2 days at 37° C. with or without SWNTs dispersed in the medium. The cells were washed and imaged by phase contrast light microscopy. FIG. 5A, no SWNTs. FIG. 5B, the medium contained SWNTs (approximately 100 μg/ml) dispersed with bovine serum albumin. There was no obvious accumulation of SWNTs in the cells. In FIG. 5C, the medium contained SWNTs (approximately 5 μg/ml) dispersed with PEDOT/PSS. Despite having a concentration of SWNTs that was 20 times lower than those dispersed with bovine serum albumin, there was unexpected and obvious accumulation of dark material (SWNTs) within vesicles in the perinuclear region of cells. These vesicles were shown to contain SWNTs by micro Raman analysis (see FIGS. 6A and 6B).

FIGS. 6A to 6B show NRK cells exposed to SWNTs (approximately 5 μg/ml) dispersed in PEDOT/PSS for 2 days and live cells were examined by light microscopy. Panels A, B, and C show images of cells containing many vesicles loaded with dark material (SWNTs) taken at 20 second intervals. The arrow in each panel tracks the movement of one vesicle as it processes towards the nuclear area in the cell.

FIG. 7A shows NRK cells were exposed to SWNTs (approximately 5 μg/ml) dispersed in PEDOT/PSS for 2 days. The cells were then fixed with paraformaldehyde and analyzed by micro Raman spectroscopy. The Raman data was collected with a 633 nm laser using a 50× objective with and average of 3 10 second integration times. Panel A, a picture of the cell analyzed showing the location of the vesicle on which the laser was focused (black circle). FIG. 7B shows a Raman spectra from the vesicle showing the distinctive SWNT Raman signature, especially the strong “G” band at ˜1,590 cm⁻¹. These data demonstrate that PEDOT/PSS dispersed SWNTs are present in the intracellular vesicles.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method of intra and extracellular delivery of compositions to cells comprising:
contacting one or more cells with a compositions comprising a water-soluble rod-coil polymer, wherein the water-soluble rod-coil polymer comprises an agent for delivery to the cell about a carbon nanostructure, wherein the water-soluble rod-coil polymer is coated with a water-soluble, low band gap conductive polymer.

2. The method of claim 1, wherein the carbon nanostructure comprises a carbon nanotube.

3. The method of claim 1, wherein the agent is within the carbon nanostructure.

4. The method of claim 1, wherein the agent is water-insoluble.

5. The method of claim 1, wherein the agent is susceptible to degradation by an organism when administered systemically.

6. The method of claim 1, wherein the agent is highly toxic to the host when administered systemically.

7. The method of claim 1, wherein the water-soluble rod-coil polymer has a solubility of greater than 20 times that of the non-coated carbon nanostructure.

8. The method of claim 1, wherein the intracellular uptake of the water-soluble rod-coil polymer is measured with normal rat kidney cells, and the water-soluble rod-coil polymer achieves the same level of intracellular location as non-polymer coated carbon nanostructure at less than one twentieth the concentration of water-soluble rod-coil polymers.

9. The method of claim 1, further comprising the step of contacting the water-soluble rod-coil polymer with cells, wherein the water-soluble rod-coil polymer is capable of intracellular transport at a concentration at least 10 times lower that carbon nanostructures that are non-polymer coated.

10. The method of claim 1, further comprising the step of contacting the water-soluble rod-coil polymer with cells, wherein the water-soluble rod-coil polymer is capable of intracellular transport at a concentration at least 10 times lower that carbon nanostructures that are non-polymer coated and disrupting the water-soluble rod-coil polymer within the cells to deliver the agent.

11. The method of claim 1, wherein the carbon nanostructure comprises carbon nanotubes that are coated with poly(3,4-ethylenedioxythiophene) poly(styrenesulfonate) (PEDOT/PSS).

12. A water-soluble conjugated system comprising:
a water-soluble rod-coil polymer comprising a carbon nanostructure coated with a water-soluble, low band gap conductive polymers selected from polypyrroles (PPy), polythiophene (PT), poly(3,4-ethylenedioxythiophene) poly(styrenesulfonate) (PEDOT/PSS); water-soluble low band gap polymer such as: poly(thieno[3,4-b]thiophenes), poly(thienylene vinylenes), hydroxyl-capped polyfluorenes (PFOH), cyclopentadithiophenes (CPDT) and oligomers and alternating copolymers thereof, ionic water-soluble polymers (cationic or anionic), polyfluorenes (PF), alternating polyfluorenylene ethynylene (PFE) copolymers comprising alternating fluorene and phenylene-oxadiazole-phenylene comprising alternating fluorene and 1,4-phenylene; water-soluble linear polyindolquinones; water-soluble poly(p-phenylenes) (PPP); water-soluble poly(p-phenylene vinylene) (PPV); water-soluble polypyridines; water-soluble ionic polyacetylene; and a biologically active agent disposed about the carbon nanostructure and the polymer.

13. The system of claim 12, wherein the polymer is selected from poly(thienylene vinylenes), cyclopentadithiophenes (CPDT), polyfluorenes (PF), polyfluorenylene ethynylene (PFE), phenylene-oxadiazole-phenylene, Polyindolquinones, and polypyridines.

14. The system of claim 12, wherein the polymer is selected from polypyrroles (PPy), polythiophene (PT), poly(3,4-ethylenedioxythiophene) poly(styrenesulfonate) (PEDOT/PSS), poly(thieno[3,4-b]thiophenes), hydroxyl-capped polyfluorenes (PFOH), 1,4-phenylene, poly(p-phenylenes) (PPP), poly(p-phenylene vinylene) (PPV) and polyacetylene.

15. The system of claim 12, wherein the carbon nanostructure comprises carbon nanotubes that are further functionalized through the addition of a targeting moiety.

16. The system of claim 12, wherein the carbon nanostructure comprises carbon nanotubes further comprising a targeting moiety selected from antibody, aptamer, peptide, protein, carbohydrate, lipid, nucleic acid, folate, folate receptor or a small molecule.

17. The system of claim 12, wherein the carbon nanostructure further comprise a liposome surrounding the water-soluble rod-coil polymer.

18. The system of claim 12, wherein the carbon nanostructure comprises carbon nanotubes coated with poly(3,4-ethylenedioxythiophene) poly(styrenesulfonate) (PEDOT/PSS) and the water-soluble rod-coil polymer is capable of intracellular transport at a concentration at least 10 times lower that carbon nanostructures that are not coated with a polymer.

19. The system of claim 12, wherein the water-soluble rod-coil polymer are targeted to the nucleus of cells.

20. The system of claim 12, wherein the water-soluble rod-coil polymer are targeted to the nucleus of cells and comprises an agent that modulates gene expression.

21. The system of claim 12, wherein the water-soluble rod-coil polymer is targeted to the cytoplasm of cells and comprises an agent that modulates cellular function.

22. The system of claim 12, wherein the water-soluble rod-coil polymer is disrupted using a radiofrequency field targeted to the carbon nanostructure.

23. The system of claim 12, wherein the water-soluble rod-coil polymer is disrupted using an infrared radiation to target the carbon nanostructure.

24. The system of claim 12, wherein the water-soluble rod-coil polymer is disrupted to release the agent and the agent is capable of killing the cell.

25. The system of claim 12, wherein the water-soluble rod-coil polymer comprises one or more agents that are highly toxic, degraded by the host or are provided as a reservoir for controlled release by an external source of radiation.

26. A method of killing target cells comprising:
contacting cells with a water-soluble rod-coil polymer comprising a carbon nanostructure coated with a water-soluble, low band gap conductive polymer under conditions in which the cells bind or internalize the water-soluble rod-coil polymer; and
exposing the target cells with the water-soluble rod-coil polymer with sufficient near infrared or radiofrequency radiation to kill the cell by hyperthermia.
